(19) United States
(12) Patent Application Publication
(10) Pub. No.: US 2010/0112102 A1
(43) Pub. Date: May 6, 2010

(54) THERAPEUTIC COMPOSITIONS FOR THE TREATMENT OF BENIGH PROSTATE HYPERPLASIA, PROSTATITIS, IMPOTENCE, INFERTILITY AND PROSTATE CANCER AND A METHOD FOR THE USE THEREOF

(76) Inventor: Nikolay Evgenievich Chernobayev, Tosno (RU)

Correspondence Address: Aleksandr Smushkovich, POB 140505, Brooklyn, NY, 11214, US

(21) Appl. No.: 12/450,595

(22) PCT Filed: Mar. 14, 2008
(86) PCT No.: PCT/RU2008/000164

§ 371 Date: Sep. 30, 2009

(30) Foreign Application Priority Data
May 3, 2007 (RU) 2007117858

(51) Int. Cl. A61K 36/54; A61P 35/00

(52) U.S. Cl. 424/739

(57) ABSTRACT

Therapeutic compositions are proposed for treating and preventing benign prostatic hyperplasia, prostatitis, impotency, infertility and prostate cancer and to a method for the use thereof. They are based on dextrorotary or levogeratory or racemic camphor in the form of a fatty-oil-dimexide emulsion, an oil-aqueous emulsion, or suppositories, the camphor quantity being of 0.1-15.0% by volume. The compositions are rectally administrable once a day by course of therapy up to 20 days every year. They increase the resistance of tissue colloids, activate the erection center of the parasympathetic nervous system, exhibit M- and H-cholinolytic action, produce tonic action on the ejaculation center, normalize the capillary and venule tonus, stimulate vasomotor centers of the spinal chord, enhance the synthesis of macroergic phosphates, inhibit ATP-acid splitting in the hypoxia conditions and exhibit analgesic, bactericidal and fibrinolytic effect, etc. The compositions and method enable carrying out the preventive treatment of the above-listed disease.

### CROSS-REFERENCE TO RELATED APPLICATIONS

This application is a U.S. national stage application of a PCT application PCT/RU2008/000164 filed on 14 Mar. 2008, published as WO/2008/136705, whose disclosure is incorporated herein in its entirety by reference, which PCT application claims priority of a Russian Federation application RU2007117858 filed on 3 May 2007.

### FIELD OF THE INVENTION

The present invention relates to the pharmaceutical industry and medicine.

### BACKGROUND OF THE INVENTION

Over the recent decade, different groups of preparations were provided for the treatment and prevention of benign prostate hyperplasia (designated as BPH hereinafter), prostatitis, impotence, infertility, and prostate cancer including, first of all, androgens, such as Sustanon, Omnodren, methyl testosterone, Testobromlecith, and testosterone propionate. Experience shows that these preparations neither decrease nor delay further prostate adenoma growth (please see Reference 1 at the end of description).

On the other hand, estrogens including Synestrol and Estradurin were used. The application of female sex hormones results in the involution of normal prostate glandular elements rather than of prostate adenoma (Reference 2).

At present, urologists in many countries have turned to treat BPH using selective α1-adrenoblockers including Dalfaz, Omnic, Cardura, etc, which block α1-adrenoreceptors of bladder cervix, which is the prostatic segment of the urethra. This successfully eliminates of alleviates the dynamic component of obstruction and restores the coordinated function of detrusor and bladder sphincter and thus helps to normalize urination (Reference 4). However, this treatment fails to reduce prostate size (Reference 4). Life-long consumption of the preparations is recommended.

The use of 5α-reductase blocker preparations (Proscar, Finasteride, and MSD) is based on the blockage of testosterone conversion into dehydrotestosterone. However, to achieve symptomatic improvement, such preparations must be taken for not less than one year. In one group of patients treated with Finasteride, the maximal rate of urination increased by 32.5%. The J-PSS parameters and prostate size decreased on average by 20% and 32%, respectively.

In the recent time, plant-derived preparations have been gaining increasing popularity. Two groups of such preparations are distinguished. The first group includes the preparations based on the lipid-sterol extract from the root of the tree Pygeum africanum. Widely known are the preparations Trianol, Tadenan, Prostamic. Their mechanism of action is unclear. The second group includes the preparations from the fan-leaved palm Serenoa repens (Serpens and Permixon). Their effect is based on reduced prostaglandin synthesis (Reference 5).

The above preparations have side-effects. For example, α1-adrenoblockers cause orthostatic responses, vertigo, headache, fatigue, indisposition, edema, asthenia, drowsiness, nausea, rhinitis, and retrograde ejaculation. The use of female sex hormones may be associated with toxic liver damage, overt feminization (compromised sex function, breast swelling, nipple pigmentation, testicular involution etc.). The most significant side-effects of the use of 5α-reductase blockers include impotence, reduced libido, and decreased amount of ejaculate.

Among the other methods, besides drug therapy, that are used at present to treat BPH, of special note are the approaches based on the application of thermal energy, such as hyperthermia, thermotherapy, and thermal ablation. These methods produce mainly symptomatic effects and do not influence the mechanical component of obstruction.

Modern surgical approaches to BPH treatment (radical adenomectomy, transurethral endoscopic surgery, laser surgery approaches, balloon dilatation, and permanent or temporary stents) are associated with the risk of short-term and long-term complications. Moreover, surgery is contraindicated for a large proportion of BPH patients.

To treat the inflammatory processes in the prostate, a variety of drugs are used including antibiotics, sulfonyl amides, androgens, enzymes, plant-derived preparations, as well as physiotherapy, therapeutic exercises, and spa treatment.

The conventional methods for the treatment of prostatitis are usually inefficient. One of the reasons of the low efficiency of treating BPH patients is the insufficient entry of antibacterial drugs into the prostate, so a variety of administration methods have been suggested including rectal phonophoresis, suppositories, direct administration to the prostate or to the paraprostatic adipose tissue, and others (Reference 6).

To treat erectile dysfunctions, phosphodiesterase inhibitor preparations (Viagra, Cialis, and Levitra) are used. A special position is occupied by intracavernous administration of vasoactive preparations, such as Caverject and papaverine. Long known are local vacuum decompression and different methods of surgery and prosthetics.

The drugs Viagra, Cialis, and Levitra have many contraindications and side-effects, such as asthenia, abdominal pain, back pain, diarrhea, nausea, muscular and articular pain, faints, sleeplessness, pharyngitis, rhinitis, apnea, vision disturbances, prostate dysfunctions, irregular heartbeat, tachycardia, vomiting, etc. The most significant side effects of the Caverject type preparations are psychoses, seizures, bleeding, and priapism. At present, there is no experience of using these approaches in patients younger than 18 years and older than 75 years. Surgery and prosthetics are inefficient. The surgical and prosthetic approaches are nonphysiological.

To treat patients suffering from pathological conditions of their semen, different therapeutic drugs are used including Bromocryptin, chorionic gonadotropin, testosterone, antibiotics, etc. There are proponent of each of the drugs, however, none of them is rigorously evidence-based and supported by data usually required to evaluate drug potency.

BPH is long known to be widely spread in the world and to be the most prevalent disorder of the genitourinary tract in elderly mails, which develops as early as at the age of 40-50. After 80 years of age, BPH is found in 95.5% of mails. This disorder is relatively rare only in Africans, Chinese, and Japanese, which prompted an investigation into the causes of such peculiar geographic distribution (Reference 1). The prior art most relevant to the present invention and the use thereof is disclosed in RF Patent No 2140265 (Reference 10).

### AIMS AND BRIEF DESCRIPTION OF THE PRESENT INVENTION

The objective of the present invention is to provide therapeutic compositions for the treatment and prevention of BPH, prostatitis, impotence, infertility, and prostate cancer using camphora preparations, which are able to promptly and significantly reduce the size of the prostate and to alleviate the dynamic component of obstruction and are efficient in the treatment of prostatitis, infertility, and impotence.

The above objective is attained with different camphora (dextrorotatory (D-), levorotatory (L-), and racemic (DL)) compositions, predominantly in the form of fat, oil, and Dimexide emulsion (Em. Camphorae Axungio-Oleo-Dimexidosa), oil and water emulsion (Emulsio Camphorae Oleo-Aquosa), suppositories, and intrarectal preparations intended to enhance the hydrophilic properties of prostate biocolloids and thus to restore lymph and blood circulation in the prostate (by colloid protecting mechanism).

The qualitative parameter for characterization of the protective action of camphora is the minimal amount of dry matter prevented from coagulation (the so-called “golden number”) at the dose of 0.1-15.0.

For the purposes thereof it is suggested:

The therapeutic compositions of the present invention for the treatment and prevention of benign prostate hyperplasia, prostatitis, impotence, infertility, and prostate cancer, which incorporate camphora, are characterized by that they are manufactured as fat, oil and Dimexide emulsions (Em. Camphorae Axungio-Oleo-Dimexidosa, or oil and water emulsions (Emulsio Camphorae Oleo-Aquosa), or suppositories (Supp. Camphorae) and contain the following components, their proportions indicated as % (v/v): 0.1% to 15.0% camphora in Em. Camphorae Axungio-Oleo-Dimexidosa or 0.1% to 15.0% camphora in of Emulsio Camphorae Oleo-Aquosa, or 0.1% to 15.0% Supp. Camphorae.

One embodiment of the therapeutic compositions of the present invention is characterized by that the fat, oil, and Dimexide emulsion of dextrorotatory (D-) camphora has the following proportion of its components (% v/v):

| | |
|---|---|
| Camphora | 0.1-15.0 |
| Porcine fat | 0.01-70 |
| Dimexide | 2-12 |
| Sunflower oil | the rest of the formulation |

Another embodiment of the therapeutic compositions of the present invention is characterized by that the fat, oil, and Dimexide emulsion of levorotatory (L-) camphora has the following proportion of its components (% v/v):

| | |
|---|---|
| Camphora | 0.1-15.0 |
| Porcine fat | 0.01-70 |
| Dimexide | 2-12 |
| Sunflower oil | the rest of the formulation |

Yet another embodiment of the therapeutic compositions of the present invention is characterized by that the fat, oil, and Dimexide emulsion of racemic (DL-) camphora has the following proportion of its components (% v/v):

| | |
|---|---|
| Camphora | 0.1-15.0 |
| Porcine fat | 0.01-70 |
| Dimexide | 2-12 |
| Sunflower oil | the rest of the formulation |

Still another embodiment of the therapeutic compositions of the present invention is characterized by that the fat, oil, and Dimexide emulsion with the proportion of dextrorotatory D-camphora optionally being 0.1% to 99.9% and the proportion of levorotatory L-camphora optionally being 0.1% to 99.9% has the following proportion of its components (% v/v):

| | |
|---|---|
| Camphora | 0.1-15.0 |
| Porcine fat | 0.01-70 |
| Dimexide | 2-12 |
| Sunflower oil | the rest of the formulation |

One embodiment of the therapeutic compositions of the present invention is characterized by that the oil and water emulsion of dextrorotatory D-camphora has the following proportion of its components (% v/v):

| | |
|---|---|
| Camphora | 0.1-15.0 |
| Sunflower oil | 20-85% |
| Distilled water | the rest of the formulation. |

Another embodiment of the therapeutic compositions of the present invention is characterized by that the oil and water emulsion of levorotatory L-camphora has the following proportion of its components (% v/v):

| | |
|---|---|
| Camphora | 0.1-15.0 |
| Sunflower oil | 20-85% |
| Distilled water | the rest of the formulation. |

Yet another embodiment of the therapeutic compositions of the present invention is characterized by that the oil and water emulsion of racemic DL-camphora has the following proportion of its components (% v/v):

| | |
|---|---|
| Camphora | 0.1-15.0 |
| Sunflower oil | 20-85% |
| Distilled water | the rest of the formulation. |

Still another embodiment of the therapeutic compositions of the present invention is characterized by that the oil in water emulsion of racemic DL-camphora with the proportion of dextrorotatory D-camphora optionally being 0.1% to 99.9% and the proportion of levorotatory L-camphora optionally being 0.1% to 99.9% has the following proportion of its components (% v/v):

| | |
|---|---|
| Camphora | 0.1-15.0 |
| Sunflower oil | 20-85% |
| Distilled water | the rest of the formulation. |

One embodiment of the therapeutic compositions of the present invention is characterized by that the rectal suppositories containing dextrorotatory D-camphora have the following amounts (grams) of the components required to manufacture a single suppository:

| | |
|---|---|
| Camphora | 0.003.-0.45 |
| Fat base | 2.55-2.97 |

Another embodiment of the therapeutic compositions of the present invention is characterized by that the rectal suppositories containing levorotatory L-camphora have the following amounts (grams) of the components required to manufacture a single suppository:

| | |
|---|---|
| Camphora | 0.003.-0.45 |
| Fat base | 2.55-2.97 |

Yet another embodiment of the therapeutic compositions of the present invention is characterized by that the rectal suppositories containing racemic DL-camphora have the following amounts (grams) of the components required to manufacture a single suppository:

| | |
|---|---|
| Camphora | 0.003.-0.45 |
| Fat base | 2.55-2.97 |

Still another embodiment of the therapeutic compositions of the present invention is characterized by that the rectal suppositories containing racemic (DL-) camphora with the proportion of dextrorotatory D-camphora optionally being 0.1% to 99.9% and the proportion of levorotatory L-camphora optionally being 0.1% to 99.9% have the following amounts (gram) of the components required to manufacture a single suppository:

| | |
|---|---|
| Camphora | 0.003.-0.45 |
| Fat base | 2.55-2.97 |

One embodiment of the therapeutic compositions of the present invention for treating and prevention of benign prostate hyperplasia, prostatitis, impotence, infertility, and prostate cancer, which comprise camphora, is characterized by that the fat, oil, and Dimexide emulsion of camphora is supplemented with fir and eucalyptus oils and has the following proportion of its components (% v/v):

| | |
|---|---|
| Camphora | 0.1-15.0 |
| Firry oil | 0.1-3.0 |
| Eucalyptus oil | 0.1-2.0 |
| Porcine fat | 0.01-70 |
| Dimexide | 2-12 |
| Sunflower oil | the rest of the formulation. |

One embodiment of the therapeutic compositions of the present invention is characterized by that the fat-oil-Dimexide emulsion of dextrorotatory D-camphora supplemented with fir and eucalyptus oils has the following proportion of its components (% v/v):

| | |
|---|---|
| Camphora | 0.1-15.0 |
| Firry oil | 0.1-3.0 |
| Eucalyptus oil | 0.1-2.0 |
| Porcine fat | 0.01-70 |
| Dimexide | 2-12 |
| Sunflower oil | the rest of the formulation. |

Another embodiment of the therapeutic compositions of the present invention is characterized by that the fat, oil, and Dimexide emulsion of levorotatory L-camphora supplemented with fir and eucalyptus oils has the following proportion of its components (% v/v):

| | |
|---|---|
| Camphora | 0.1-15.0 |
| Firry oil | 0.1-3.0 |
| Eucalyptus oil | 0.1-2.0 |
| Porcine fat | 0.01-70 |
| Dimexide | 2-12 |
| Sunflower oil | the rest of the formulation. |

Yet another embodiment of the therapeutic compositions of the present invention is characterized by that the fat, oil, and Dimexide emulsion of racemic DL-camphora supplemented with fir and eucalyptus oils has the following proportion of its components (% v/v):

| | |
|---|---|
| Camphora | 0.1-15.0 |
| Firry oil | 0.1-3.0 |
| Eucalyptus oil | 0.1-2.0 |
| Porcine fat | 0.01-70 |
| Dimexide | 2-12 |
| Sunflower oil | the rest of the formulation. |

Still another embodiment of the therapeutic compositions of the present invention is characterized by that the fat-oil-Dimexide emulsion of camphora with the proportion of dextrorotatory D-camphora optionally being 0.1% to 99.9% and the proportion of levorotatory L-camphora optionally being 0.1% to 99.9% supplemented with firry and eucalyptus oils has the following proportion of its components (% v/v):

| | |
|---|---|
| Camphora | 0.1-15.0 |
| Firry oil | 0.1-3.0 |
| Eucalyptus oil | 0.1-2.0 |
| Porcine fat | 0.01-70 |
| Dimexide | 2-12 |
| Sunflower oil | the rest of the formulation. |

A method of using the composition is provided, characterized in that fat-oil-dimexide emulsion of camphora and rectal suppositories are used for chronic prostatitis or prostate cancer treatment and prevention.

Another method of using the composition is characterized in that fat-oil-dimexide emulsion of camphora and rectal suppositories are used for benign prostate hyperplasia treatment and prevention.

Another method of using the composition is characterized in that fat-oil-dimexide emulsion of camphora is used for impotency or infertility treatment and prevention.

Another method of using the composition is characterized in that fat-oil-dimexide emulsion of camphora, fat-oil-dimexide emulsion of camphora supplemented with fir-tree and eucalyptus oils, oil-and-water emulsion of camphora, and rectal suppositories are used for acute prostatitis or chronic prostatitis exacerbation treatment and prevention.

### DETAIL DESCRIPTION OF PREFERRED EMBODIMENTS OF THE INVENTION

While the invention may be susceptible to embodiment in different forms, there are described in detail herein, specific embodiments of the present invention, with the understanding that the present disclosure is to be considered an exemplification of the principles of the invention, and is not intended to limit the invention to that as exemplified herein.

The therapeutic compositions of the present invention are illustrated with the examples (A) below. Fat, oil, and Dimexide emulsions of camphora (% v/v):

### Example 1

| | |
|---|---|
| Camphora (D-) | 2.0 |
| Porcine fat | 3.0 |
| Dimexide | 3.0 |
| Sunflower oil | the rest of the formulation. |

### Example 2

| | |
|---|---|
| Camphora (L-) | 3.0 |
| Porcine fat | 20.0 |
| Dimexide | 6.0 |
| Sunflower oil | the rest of the formulation. |

### Example 3

| | |
|---|---|
| Camphora (DL-) | 5.0 |
| Porcine fat | 50.0 |
| Dimexide | 10.0 |
| Sunflower oil | the rest of the formulation. |

### Example 4

| | |
|---|---|
| Camphora (20% D-, 80% L-) | 1.2 |
| Porcine fat | 54.0 |
| Dimexide | 7.5 |
| Sunflower oil | the rest of the formulation. |

### Example 5

| | |
|---|---|
| Camphora (90% D-, 10% L-) | 1.2 |
| Porcine fat | 25.0 |
| Dimexide | 1.2 |
| Sunflower oil | the rest of the formulation. |

### Examples (B)

### Oil and Water Emulsions of Camphora (% v/v)

### Example 1

| | |
|---|---|
| Camphora (D-) | 2.0 |
| Sunflower oil | 30.0 |
| Distilled water | the rest of the formulation. |

### Example 2

| | |
|---|---|
| Camphora (L-) | 6.0 |
| Sunflower oil | 40.0 |
| Distilled water | the rest of the formulation. |

### Example 3

| | |
|---|---|
| Camphora (DL-) | 10.0 |
| Sunflower oil | 60.0 |
| Distilled water | the rest of the formulation. |

### Example 4

| | |
|---|---|
| Camphora (20% D-, 80% L-) | 8.0 |
| Sunflower oil | 75.0 |
| Distilled water | the rest of the formulation. |

### Example 5

| | |
|---|---|
| Camphora (70% D-, 30% L-) | 1.0 |
| Sunflower oil | 70.0 |
| Distilled water | the rest of the formulation. |

### Examples (C)

### Rectal Suppositories (the Amounts of Camphora and Fat Base in a Single Suppository, g)

### Example 1

| | |
|---|---|
| Camphora (D-) | 0.003 |
| Fat base | 2.997 |

### Example 2

| | |
|---|---|
| Camphora (L-) | 0.006 |
| Fat base | 2.994 |

### Example 3

| | |
|---|---|
| Camphora (DL-) | 0.03 |
| Fat base | 2.97 |

### Example 4

| | |
|---|---|
| Camphora (D-) | 0.01 |
| Camphora (L-) | 0.05 |
| Fat base | 2.94 |

### Example 5

| | |
|---|---|
| Camphora (D-) | 0.05 |
| Camphora (L-) | 0.02 |
| Fat base | 2.93 |

### Examples (D)

### Fat, Oil, and Dimexide Emulsions of Camphora Supplemented with Fir and Eucalyptus Oils, % v/v

### Example 1

| | |
|---|---|
| Camphora (D-) | 2.0 |
| Firry oil | 1.0 |
| Eucalyptus oil | 0.3 |
| Porcine fat | 3.0 |
| Dimexide | 3.0 |
| Sunflower oil | the rest of the preparation |

### Example 2

| | |
|---|---|
| Camphora (L-) | 3.0 |
| Firry oil | 1.5 |
| Eucalyptus oil | 0.8 |
| Porcine fat | 20.0 |
| Dimexide | 6.0 |
| Sunflower oil | the rest of the preparation |

### Example 3

| | |
|---|---|
| Camphora (DL-) | 5.0 |
| Firry oil | 2.0 |
| Eucalyptus oil | 1.5 |
| Porcine fat | 50.0 |
| Dimexide | 10.0 |
| Sunflower oil | the rest of the preparation |

### Example 4

| | |
|---|---|
| Camphora (20% D-, 80% L-) | 1.2 |
| Firry oil | 2.2 |
| Eucalyptus oil | 1.3 |
| Porcine fat | 54.0 |
| Dimexide | 7.5 |
| Sunflower oil | the rest of the preparation |

### Example 5

| | |
|---|---|
| Camphora (90% D-, 10% L-) | 4.2 |
| Firry oil | 0.4 |
| Eucalyptus oil | 0.2 |
| Porcine fat | 25.0 |
| Dimexide | 1.2 |
| Sunflower oil | the rest of the preparation |

The method of using of the therapeutic compositions of the present invention is characterized by the following: to treat and prevent chronic prostatitis or prostate cancer, camphora emulsions in fat, oil, and Dimexide and rectal suppositories are used; to treat and prevent BPH, camphora emulsions in fat, oil, and Dimexide and rectal suppositories are used; to treat and prevent impotence and infertility, camphora emulsions in fat, oil, and Dimexide are used to treat and prevent acute prostatitis or the exacerbations of chronic prostatitis, camphora emulsions in fat, oil, and Dimexide, camphora emulsions in fat, oil, and Dimexide supplemented with fir and eucalyptus oil, camphora emulsions in oil and water, and camphora rectal suppositories are used, the above therapeutic composition being administered rectally.

The examples (E) of the usage of the therapeutic compositions of the present invention are provided below.

### Example 1

BPH patients were successfully treated with the 2% camphora (dextrorotatory, D-) emulsion in fat, oil, and Dimexide of. In total, 425 patients aged 50 to 88 years have been treated. After 2 months of the treatment, on average, the combined score of clinical manifestations by J-PSS scale decreased by 17.3 (from 24.2 to 6.9). The life quality index L decreased by 3.2 (from 5.2 to 2). The urine flow index increased from 6.2 to 13.2 mL, i.e., by 7 mL. The size of the prostate decreased on average by 22.4 cm³ (according to ultrasonography).

### Example 2

Chronic prostatitis was treated with the 3% camphora (levorotatory, L-) emulsions in fat, oil, and Dimexide of. In total, 514 patients aged 20 to 51 years have been treated. The treatment lasted for 1 to 2 months. Prostate secretion normalization was evident within the first two weeks of the treatment. In the cases of bacterial prostatitis, beneficial results of bacteriological tests of the 3^{rd} portion of urine and of prostate secretion were obtained. The long-term (5 years) results of the treatment are available for 71 patients. The results are good.

### Example 3

Erectile dysfunction was successfully treated the with 5% camphora (racemic, DL-) emulsion in fat, oil, and Dimexide. The number of patients aged 21 to 91 years exceeds 6000. Good results have been obtained in 95% of the patients. One patient discontinued his treatment prematurely. All patients reported increased intercourse duration.

### Example 4

Over the last 10 years, patients with semen pathology were treated with the 1.2% camphora (20% dextrorotatory, D; 80% levorotatory, L) emulsion in fat, oil, and Dimexide. The treatment was provided to 43 patients. Thereafter, the number of pregnancies was 38.

### Example 5

After the main treatment course, BPH patients received preventive treatment with the 4.2% camphora (90% dextrorotatory, D; 10% levorotatory, L) emulsion in fat, oil, and Dimexide. The number of patients was 743. The quality of life index L did not change significantly and remained stable. Long-term results of the preventive treatment are available for 116 patients. The results are good.

### Example 6

The preventive treatment of prostate cancer with the 4.2% camphora (10% dextrorotatory, D; 90% levorotatory, L) emulsion in fat, oil, and Dimexide was provided to 21 patients. Follow-up periods ranged from 5 to 10 years. No signs of prostate cancer were found. Patient age was 52 to 73 years.

### Example 7

Acute prostatitis was treated with the 2% dextrorotatory D-camphora emulsion in oil and water at the dose of 5 mL daily. The number of patients was 15. The clinical manifestations of the condition stabilized within 5-7 days.

### Example 8

For the preventive treatment of chronic prostatitis, the 2% racemic (DL-) camphora emulsion in fat, oil, and Dimexide was used. The number of patients treated over 1 year was 35. Long-term results (more than 3 years) are available for 12 patients. No relapses of the condition were found. Control microbiological tests of the 3^{rd} portion of urine and of prostate secretion revealed no microflora. Long-term results (more than 5 years) are available for 36 patients. The results are good.

### Example 9

For the preventive treatment of chronic prostatitis, the 3% camphora (30% dextrorotatory, D; 70% levorotatory, L) emulsion in fat, oil, and Dimexide of was used. The patients were treated for 20 days annually. The number of patients was 214. Long-term results (more than 5 years) are available for 36 patients. The results are good.

### Example 10

Rectal suppositories containing the 0.1% camphora were used to treat BPH patients. Patient number was 122. Long-term results (more than 5 years) are available for 47 patients. The quality of life index L did not change. No clinically significant BPH was found.

### Example 11

For the preventive treatment of 68 chronic prostatitis patients, the rectal suppositories with 0.2% camphora were used by 20-days courses 3-4 times annually for two years. Long-term results (more than 3 years) are available for 12 patients. The patients reported no complaints about their health. Control microscopy of prostate secretion revealed no pathology. Crystallization phenomenon was positive in 9 patients.

### Example 12

Prostate cancer prevention was attempted in 21 subjects using rectal suppositories with the 1% camphora. The subjects were followed-up for 5 to 10 years. No malignization of the already present benign prostate hyperplasia was found. Free and bound PSA levels were normal. No clinically significant enlargement of the prostate was found.

### Example 13

To treat chronic prostatitis in 14 patients aged 31 to 54 years, the 1% dextrorotatory D-camphora emulsion in fat, oil, and Dimexide was used. The patients received the treatment over a period of one to two months. The normalization of prostate secretion was evident within the first 10 days of the treatment. The results of the microbiological tests of the 3^{rd} portion of urine and of prostate secretion also normalized. Long-term results (more than 1 year) are available for 11 patients. The results are good.

### Example 14

The 1.5% racemic (DL-) camphora emulsion in fat, oil, and Dimexide supplemented with fir and eucalyptus oils was successfully used to treat BPH in 21 patients aged 54 to 62 years. After 2 months of the treatment, the mean score of clinical manifestation by J-PSS score decreased by 17.8 (from 23.8 to 6.0). The quality of life index L deceased by 2.8 (from 3.8 to 1.0). Urine flow rate index increased from 8.9. to 15.1, i.e., by 6.2. Prostate size decreased by 15.4 cm³ on average (according to ultrasonography).

### Example 15

The 3% camphora (80% D, 20% L) emulsion in fat, oil, and Dimexide supplemented with fir and eucalyptus oils was successfully used to treat erectile dysfunction in 20 patients aged 41 to 47 years. The nocturnal erection test became positive during the treatment in all who was examined. The anal and bulbocavernous reflexes upon control examination performed after one month were positive.

### Example 16

The 2% levorotatory (L-) camphora emulsion in fat, oil, and Dimexide supplemented with fir and eucalyptus oils was successfully used to prevent the exacerbations of chronic prostatitis in 13 patients treated for 1 year. No microflora growth was found upon microbiological testing of the 3^{rd} portion of urine and of prostate secretion. The results of microscopy of prostate secretion in 8 patients were normal during 1 year.

The above examples of different formulations of camphora (fat, oil, and Dimexide emulsion, oil and water emulsion, and suppositories with camphora) having different properties (dextrorotatory, D-; levorotatory, L-; racemic, DL-, with different proportion of the dextrorotatory D- and levorotatory L-form) and at different percent levels prove the efficiency of the therapeutic compositions of the present invention and of the therapeutic use thereof.

The protective effect of the lyophilic colloid of camphora is produced on the biocolloids of the ground substance of the stromal tissue and secretion of the prostate, which, because of the changes that occur with increasing age and upon inflammation, acquire hydrophobic properties associated with increased rigidity. This leads to alterations in blood flow and lymph circulation and to the reduction of lymphatic roots. The therapeutic compositions of the present invention increase the resistance of tissue colloids and enhance their hydrophilic properties thus increasing the dispersity of proteins (enzymes and hormones).

In medicine, camphora is used due to its antiseptic effect, to which pyogenic microbes are most sensitive. Camphora is partly excreted unchanged by glands and thus facilitates the liquefaction of their secretions (Reference 8). Camphora influences the metabolism of macroergic phosphates, so ATP depletion under hypoxia is inhibited, which is explained by enhanced resynthesis of macroergic phosphates because of the activation of anaerobic glycolysis (Reference 8). It is generally believed that when the level of AMP acid decreases, spermatozoa lose their motility. The stimulatory effects of camphora on the vasomotor centers of the spine are known. Camphora may change vascular tone by acting on vascular chemoreceptors. Camphora normalizes the tone of capillaries and venules and restores the compromised permeability of capillary membranes. Camphora is believed to be a reliable means to enhance venous tonus (Reference 8). The effect of camphora on venous tonus is reflectory. Camphora stimulates the reflex activity of the spinal centers of the lumbar segment of the spine or restores its activity in the cases of spinal shock, which may be interpreted as a result of the direct effects of camphora on spine centers (Reference 9). One of the important properties that define camphora pharmacodynamics is its M- and N-cholinergic activity. When camphora enters blood and lymph, it is absorbed by erythrocytes, lipoprotein complexes, proteins, foreign microbial particles, and fibrin clots thus facilitating their clearance. The starting material to obtain camphora is camphor tree (Cinnamomum camphora), which grows wild in China and Japan, and sweet basil (Ocinum canun Sims.) which grows in Africa and South China.

Fir oil contains more than 35 biologically active substances. Among them, phytoncides produce kill microbes. Fir oil activates sex glands functions by virtue of its vitamin E content (0.6% to 1.6%) and easily penetrates the skin and mucosae. Dimexide too is able to penetrate biological membranes thus realizing its specific effects including antiinflammatory, antipyretic, antiseptic, and fibrinolytic activities. Dimexide enhances drug penetration through the skin because it possesses transporting activity (7). Besides that, in the present invention, Dimexide is used as fat emulator and preservative.

The action of eucalyptus oil is mainly based on its antimicrobial and antiinflammatory effects. The therapeutic compositions of the present invention are administered rectally once a day.

The indications to treat BPH patients were clinical manifestations as reported in patient complaints (their score by J-PSS scale was 20 or more). The fat, oil, and Dimexide emulsions were mainly applied by administering them into the anus at the dose of 5 mL using a microenema once a day for 2-4 months until stabilization of clinical manifestation. The patients were examined 2-4 months thereafter. The following parameters were used to assess the effects of the treatment:

clinical manifestations by J-PSS scale;

life quality index;

prostate size; and

residual urine volume in Stage 2 patients.

More than 1121 patients aged 51 to 91 have been treated over 10 years. Among them there were 1050 Stage 1 BPH patients and 71 Stage 2 BPH patients. After 3 months, the combined score of clinical manifestations by the J-PSS score decreased by 21.2 (from 25.9 to 4.7), the quality of life index L decreased by 3.8 (from 5.5. to 1.7), the urine flow index Qₘₐₓ increased from 7.4 mL/sec to 13.2 mL/sec, i.e., by 5.8 mL/sec. Prostate size decreased on average from 24.8 cm³ (according to ultrasonography). The amount of residual urine in Stage 2 patients decreased by 134.5 cm³ (from 149.7 to 15.2 cm³). Thereafter, these patients used rectal suppositories by 20-days courses once a year. If symptoms aggravated and quality of life worsened, the treatment changed to fat, oil, and dimexide emulsion application until clinical manifestation stabilized. The long-term results of the treatment are available for 521 patients (more than 5 years) and for 21 patients (more than 10 years). Their objective data and life quality were stable.

The fat, oil, and Dimexide emulsions were successfully used to treat chronic prostatitis patients. The preparations were applied for 1-2 months once a day at the dose of 5 mL of emulsion. The treatment was provided to 4030 patients aged 20 to 51 years. The criteria of treatment quality were patient complaints, the conditions of prostate secretion, crystallization phenomenon, the results of prostate palpation and ultrasonography, and bacteriological tests of the 3^{rd} portion of urine and of prostate secretion. The patients of this group were treated for about 1-2 months. Long-term results (more than 5 years) are available for 1218 patients. The results are good. After the main treatment course, these patients used the same preparation by 20-days courses once in 3-4 years or rectal suppositories for 20 days once in 3-4 years.

In very rare cases of acute prostatitis or exacerbated chronic prostatitis, the oil and water emulsions of camphora were used for up to 7 days. Then, after the normalization of body temperature and other clinical symptoms, the fat, oil, and Dimexide emulsions of camphora were used for up to 1-2 months. Thereafter, the same emulsions or suppositories were used once in 3-4 years.

Highly dispersed camphora preparations in the form of fat, oil, and Dimexide emulsion proved to be highly efficient in treating of patients having erectile dysfunction. The number of patients was over 6000. Patients' ages were 21 to 91 years. All treatment results were positive. Only one patient discontinued treatment for unknown reasons. The parameters of treatment quality were patient's complaints and self-reports and nocturnal erection test results. All patients reported increased intercourse duration. Follow-up (more than 5 years) results for 112 patients are available. The results are excellent. Thereafter, fat, oil, and Dimexide emulsion was applied by 20-days courses once in 3-4 years. Good results have been also obtained in treating of Stages 2 and 3 oligospermia patients. Over the last 10 years, 43 patients were treated. The number of pregnancies was 38, the treatment regimen being 5 mL of the fat, oil, and Dimexide emulsion of camphora applied daily by night for 2 to 6 months.

The camphora preparations proved to be efficient upon rectal application have the following pharmacological properties:
show colloid protecting activity towards prostatic secretion biocolloids and stromal tissue;
possess M- and N-cholinergic activity;
antagonize capillary poisons;
produce the tonic effect on the erection center located in the sacral segment of the parasympathetic nervous system;
produce the tonic effect on the ejaculation center of the sympathetic nervous system;
normalize the tone of venules and capillaries;
stimulate the vasomotor centers of the central nervous system;
enhance macroergic phosphates synthesis and inhibit ATP depletion under hypoxic conditions; and
produce analgesic, bactericidal, and fibrinolytic effects. Thus, the use of the therapeutic compositions of camphora according to the present invention will provide the following:
the expansion of the range of preparations and therapeutic and preventive approaches used to treat patients suffering from BPH, prostatitis, infertility, impotence, and prostate cancer, and
the possibility to provide the preventive treatment of the above conditions for many decades.

### REFERENCES

(1) Tiktinskiy O. L., Novikov I. F, and Mikhailichenko V. V. Adenoma of the prostate (paraurethral gland). In: Zabolevaniya Polovykh Organov u Muzhchin (Sex Organ Diseases in Mails). Moscow: Meditsyna, 1985, pp. 119-148 (in Russian.).
(2) Shabad L. M. Some principal aspects of pathologic anatomy and pathogenesis of adenomatous prostate lesions and prostate cancer. In: Voprosy Prakticheskoy Urologii (Issues of Practical Urology). Moscow, 1949, pp. 102-105. (in Russian.).
(3) Loran O. B. and Vishnevskiy A. Ye. Klinicheskaya Farmakologiya i Terapiya, 1997, No. 1, pp. 87-89 (In Russian).
(4) Tkachuk V. N. and Kuzmin I. V. The efficiency of Alfuosine (Dalfaz) in benign prostate hyperplasia patients. Urologiya i Nefrologiya, 1998, No 2, pp. 38-40. (In Russian)
(5) Loran O. B. On the possibility of drug therapy for benign hyperplasia of prostate. TOP Meditzyna, 1997, No 3, pp. 8-10. (in Russian.).
(6) Seksopatologiya (Handbook of Sexual Pathology). Ed. by G. S. Vasilchenko. Moscow: Meditsyna, 1990, 529 pp. (in Russian.).
(7) Borzhiyevskiy Ts. K. and Fityo I. S. The use Dimexide-and-drug mixtures in the treatment of chronic prostatitis. Urologiya i Nefrologiya, 1980, No 2, pp. 24-36. (in Russian.).
(8) Saratikov A. S. Camphora: Its Pharmacology and Clinical Use. Tomsk: Izdatelstvo Tomskogo Universiteta, 1996. (in Russian.).
(9) Dogayeva I. Ye. Camphora shock and its pathogenesis. Dissertation Theses. Irkutsk, 1954. (in Russian.).
(10) RF Patent No 2140265, 1999.

1. Therapeutic compositions for the treatment and prevention of benign prostate hyperplasia, prostatitis, impotence, infertility, and prostate cancer, said compositions are produced at least in one of the following forms:
(A) Dimexide emulsions (Em. Camphorae Axungio-Oleo-Dimexidosa) including fat, oil and Dimexide, or
(B) emulsions (Emulsio Camphorae Oleo-Aquosa) including oil and water, or
(C) suppositories (Supp. Camphorae);
and said compositions include from 0.1% to 0.15.0% (v/v) of Camphora.

2. The therapeutic composition of said form (A) according to claim 1, including the following components in the proportion of (% v/v):

| | |
|---|---|
| Camphora | 0.1-15.0 |
| Porcine fat | 0.01-70 |
| Dimexide | 2-12 |
| Sunflower oil | the rest of said composition |

3. The therapeutic composition of said form (A) according to claim 1, characterized in that said Camphora is represented by the levorotatory (L-) Camphora, and said composition includes the following components in the proportion of (% v/v):

| | |
|---|---|
| Camphora | 0.1-15.0 |
| Porcine fat | 0.01-70 |
| Dimexide | 2-12 |
| Sunflower oil | the rest of said composition |

4. The therapeutic composition of said form (A) according to claim 1 characterized in that said Camphora is represented by the racemic (DL-) Camphora and said composition includes the following components in the proportion of (% v/v):

| | |
|---|---|
| Camphora | 0.1-15.0 |
| Porcine fat | 0.01-70 |
| Dimexide | 2-12 |
| Sunflower oil | the rest of said composition |

5. The therapeutic composition of said form (A) according to claim 1, characterized in that said Camphora is represented by a combination of the dextrorotatory D-Camphora, having the fraction from 0.1% to 99.9%, and the levorotatory L-Camphora, having the fraction from 0.1% to 99.9%, and said composition includes the following components in the proportion of (% v/v):

| | |
|---|---|
| Camphora | 0.1-15.0 |
| Porcine fat | 0.01-70 |
| Dimexide | 2-12 |
| Sunflower oil | the rest of said composition |

6. The therapeutic composition of said form (B) according to claim 1, characterized in that said Camphora is represented by the dextrorotatory (D-) Camphora, and said composition includes the following components in the proportion of (% v/v):

| | |
|---|---|
| Camphora | 0.1-15.0 |
| Sunflower oil | 20-85% |
| Distilled water | the rest of said composition |

7. The therapeutic composition of said form (B) according to claim 1, characterized in that said Camphora is represented by the levorotatory (L-) Camphora, and said composition includes the following components in the proportion of (% v/v):

| | |
|---|---|
| Camphora | 0.1-15.0 |
| Sunflower oil | 20-85% |
| Distilled water | the rest of said composition |

8. The therapeutic composition of said form (B) according to claim 1, characterized in that said Camphora is represented by the racemic (DL-) Camphora, and said composition includes the following components in the proportion of (% v/v):

| | |
|---|---|
| Camphora | 0.1-15.0 |
| Sunflower oil | 20-85% |
| Distilled water | the rest of said composition |

9. The therapeutic composition of said form (B) according to claim 1, characterized in said Camphora is represented by the racemic (DL-) Camphora with a fraction of the dextrorotatory D-Camphora being from 0.1% to 99.9% and a fraction of the levorotatory L-Camphora being from 0.1% to 99.9%, and said composition includes the following components in the following proportion of (% v/v):

| | |
|---|---|
| Camphora | 0.1-15.0 |
| Sunflower oil | 20-85% |
| Distilled water | the rest of said composition |

10. The therapeutic composition of said form (C) according to claim 1, characterized in that said Camphora is represented by the dextrorotatory (D-) Camphora, and said composition includes the following components in the following amounts (grams) to manufacture a single rectal suppository:

| | |
|---|---|
| Camphora | 0.003.-0.45 |
| Fat base | 2.55-2.97. |

11. The therapeutic composition of said form (C) according to claim 1, characterized in that said Camphora is represented by the levorotatory (D-) Camphora and said composition includes the following components in the following amounts (grams) to manufacture a single rectal suppository:

| | |
|---|---|
| Camphora | 0.003.-0.45 |
| Fat base | 2.55-2.97. |

12. The therapeutic composition of said form (C) according to claim 1 characterized in that the rectal suppositories comprise racemic (DL-) Camphora and said composition includes the following components in the following amounts (grams) to manufacture a single rectal suppository:

| | |
|---|---|
| Camphora | 0.003.-0.45 |
| Fat base | 2.55-2.97. |

13. The therapeutic composition of said form (C) according to claim 1, characterized in that the rectal suppositories comprise racemic (DL-) Camphora with a fraction of the dextrorotatory D-Camphora being from 0.1% to 99.9% and a fraction of the levorotatory L-Camphora being from 0.1% to 99.9%, and said composition includes the following components in the following amounts (grams) to manufacture a single rectal suppository:

| | |
|---|---|
| Camphora | 0.003.-0.45 |
| Fat base | 2.55-2.97. |

14. A therapeutic composition for the treatment and prevention of benign prostate hyperplasia, prostatitis, impotence, infertility, and prostate cancer, said composition includes the following components: fat, oil, Dimexide emulsion of Camphora, firry oil, and eucalyptus oil; and has the following proportion of said components (% v/v):

| | |
|---|---|
| Camphora | 0.1-15.0 |
| Firry oil | 0.1-3.0 |
| Eucalyptus oil | 0.1-2.0 |
| Porcine fat | 0.01-70 |
| Dimexide | 2-12 |
| Sunflower oil | the rest of said composition |

15. The therapeutic composition according to claim 14 characterized in that said Dimexide emulsion of Camphora is represented by the Dimexide emulsion of dextrorotatory (D-) Camphora.

16. The therapeutic composition according to claim 14 characterized in that said Dimexide emulsion of Camphora is represented by the Dimexide emulsion of levorotatory (L-) Camphora.

17. The therapeutic composition according to claim 14 characterized in that said Dimexide emulsion of Camphora is represented by the Dimexide emulsion of racemic (DL-) Camphora.

18. The therapeutic composition according to claim 14 characterized in that said Dimexide emulsion of Camphora is represented by: (a) a fraction of the Dimexide emulsion of dextrorotatory D-Camphora being 0.1% to 99.9%, and (b) a fraction of the Dimexide emulsion of levorotatory L-Camphora being 0.1% to 99.9%.

19. A method for using the therapeutic composition according to claim 1, wherein said composition includes fat, oil, the Dimexide emulsion of Camphora, and the rectal suppositories; and said composition is used to treat and prevent chronic prostatitis and prostate cancer.

20. A method for using of the therapeutic composition according to claim 1, wherein said composition includes fat, oil, the Dimexide emulsion of Camphora, and rectal suppositories; and said composition is used to treat and prevent benign prostate hyperplasia.

21. A method for using of the therapeutic composition according to claim 1, wherein said composition includes fat, oil, and the Dimexide emulsion of Camphora; and said composition is used to treat and prevent impotence and infertility.

22. A method for using of the therapeutic composition according to claim 1, characterized in that a combination of fat, oil, and Dimexide emulsion of Camphora, a combination of fat and water emulsion of Camphora, a combination of fat, oil, and Dimexide emulsion of Camphora are supplemented with fir and eucalyptus oils; and said combinations with the rectal suppositories are used to treat and prevent acute prostatitis and the exacerbations of chronic prostatitis.

23. The method according to claim 19, characterized in that the therapeutic composition is administered rectally.

24. The method according to claim 20 characterized in that the therapeutic composition is administered rectally.

25. The method according to claim 21 characterized in that the therapeutic composition is administered rectally.

26. The method according to claim 22 characterized in that the therapeutic composition is administered rectally.
